(19) United States
(12) Patent Application Publication
(10) Pub. No.: US 2015/0038810 A1
(43) Pub. Date: Feb. 5, 2015

(54) SENSORS FOR PHOTOPLETHYSMOGRAPHY AT THE OPHTHALMIC ARTERY REGION

(71) Applicant: Richard J. Melker, Gainesville, FL (US)

(72) Inventor: Richard J. Melker, Gainesville, FL (US)

(21) Appl. No.: 14/451,909

(22) Filed: Aug. 5, 2014

Related U.S. Application Data
(60) Provisional application No. 61/862,302, filed on Aug. 5, 2013.

(51) Int. Cl. A61B 5/0295; A61B 5/08; A61B 5/00; A61B 5/1455

(52) U.S. Cl. CPC A61B 5/0295; A61B 5/14552; A61B 5/0806; A61B 5/6819; A61B 5/6821
USPC 600/323; 600/481; 600/507; 600/529

(57) ABSTRACT

Provided according to embodiments of the invention are methods for monitoring an individual including securing a photoplethysmography (PPG) sensor comprising at least one emitter and at least one detector onto skin overlying an ophthalmic artery region of the individual; and obtaining PPG signals generated by the PPG sensor. Sensors for photoplethysmographic monitoring at the ophthalmic artery region are also provided.

### CROSS-REFERENCE TO RELATED APPLICATIONS

This application claims the benefit of U.S. Provisional Application No. 61/862,302, filed Aug. 5, 2013, the contents of which are incorporated herein by reference in their entirety.

### FIELD OF THE INVENTION

The present invention relates to biological sensors, and in particular, to photoplethysmography sensors.

### BACKGROUND OF THE INVENTION

Photoplethysmography, or “PPG”, is an optical technique for detecting blood volume changes in a tissue. In this technique, one or more emitters are used to direct light at a tissue and one or more detectors are used to detect the light that is transmitted through the tissue (“transmissive PPG”) or reflected by the tissue (“reflectance PPG”). The volume of blood, or perfusion, of the tissue affects the amount of light that is transmitted or reflected. Thus, the PPG signal varies with changes in the perfusion of the tissue.

The blood volume in a tissue changes with each heartbeat, and so the PPG signal also varies with each heartbeat. Traditionally, this component of the PPG signal is referred to as the “AC component” component of the signal, and is also often referred to as the “pulsatile component.” Blood volume is also affected by other physiological processes in the body, including respiration, venous blood volume, sympathetic and parasympathetic tone and certain pathologies. The changes in the PPG signal due to these and other physiological processes, along with changes in the PPG signal due to noise caused by non-physiological processes such as ambient light and bodily movement, have traditionally been referred to collectively as the “DC component.”

The present inventors have recently extracted specific parameters from the DC component. Traditional sites for monitoring PPG, such as fingers and toes, generally provide a relatively small PPG signal, and the quality of this signal may be negatively impacted by sympathetic innervation in these tissue sites. Thus, the DC component signal from traditional peripheral sites may not be of sufficient strength and quality to effectively separate out the signals from different physiological processes.

Thus, new devices, systems and methods for PPG monitoring may be desirable.

### SUMMARY OF EMBODIMENTS OF THE INVENTION

Provided according to embodiments of the invention are methods for monitoring an individual that include securing a photoplethysmography (PPG) sensor that includes at least one emitter and at least one detector onto skin overlying an ophthalmic artery region of the individual; and obtaining PPG signals generated by the PPG sensor. In some embodiments, the methods further include analyzing the PPG signals to obtain at least one blood oxygen saturation measurement, analyzing the PPG signals to monitor blood flow to the brain of the individual, and/or analyzing the PPG signals to monitor respiration in the individual. In some embodiments, methods further include securing a PPG sensor onto a secondary site on the individual and obtaining PPG signals generated by the PPG sensor at the secondary site on said individual.

Also provided according to embodiments of the invention are sensors configured to secure to skin overlying the ophthalmic artery region. In some cases, the sensor is configured to secure to a nasal bridge. For example, a body portion may secure to the nasal bridge and a sensor portion may secure to skin overlying the ophthalmic artery region. In some embodiments, a portion of the sensor is secured to the skin overlying the ophthalmic artery region with an adhesive. Furthermore, in some cases, the sensor is integral or attached to an eyeglass frame.

Further provided according to embodiments of the invention are systems for PPG monitoring of an individual that include PPG sensor according to an embodiment of the invention and a computer configured to process and/or analyze signals from the PPG sensor. In some embodiments, the systems may analyze the PPG signals to obtain at least one blood oxygen saturation measurement, analyze the PPG signals to monitor blood flow to the brain of the individual, and/or analyze the PPG signals to monitor respiration in the individual.

### BRIEF DESCRIPTION OF THE DRAWINGS

The following drawings are provided to illustrate various aspects of the present inventive concept and are not intended to limit the scope of the present invention unless specified herein.

FIG. 1 provides an illustration of the ophthalmic artery region.

FIG. 2 illustrates a PPG sensor according to an embodiment of the invention that is secured to the nasal bridge.

FIG. 3 illustrates a PPG sensor according to an embodiment of the invention that includes two PPG sensor portions.

FIG. 4 illustrates a PPG sensor according to an embodiment of the invention wherein the body portion is shaped into an eyeglass frame.

FIG. 5 illustrates a PPG sensor according to an embodiment of the invention that secures with an adhesive.

FIG. 6 illustrates a PPG sensor according to an embodiment of the invention that includes a secondary respiration sensor.

### DETAILED DESCRIPTION OF EMBODIMENTS OF THE INVENTION

The present invention now will be described more fully hereinafter with reference to the accompanying drawings, in which embodiments of the invention are shown. However, this invention should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

It will be understood that when an element is referred to as being “on” or “adjacent” to another element, it can be directly on or directly adjacent to the other element or intervening elements may also be present. In contrast, when an element is referred to as being “directly on” or “directly adjacent” to another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers refer to like elements throughout the specification.

It will be understood that, although the terms first, second, etc. may be used herein to describe various elements, these elements should not be limited by these terms. These terms are only used to distinguish one element from another. Thus, a first element discussed below could be termed a second element without departing from the teachings of the present invention.

Embodiments of the present invention are described herein with reference to schematic illustrations of idealized embodiments of the present invention. As such, variations from the shapes of the illustrations as a result, for example, of manufacturing techniques and/or tolerances, are to be expected.

### Photoplethysmography Sensors

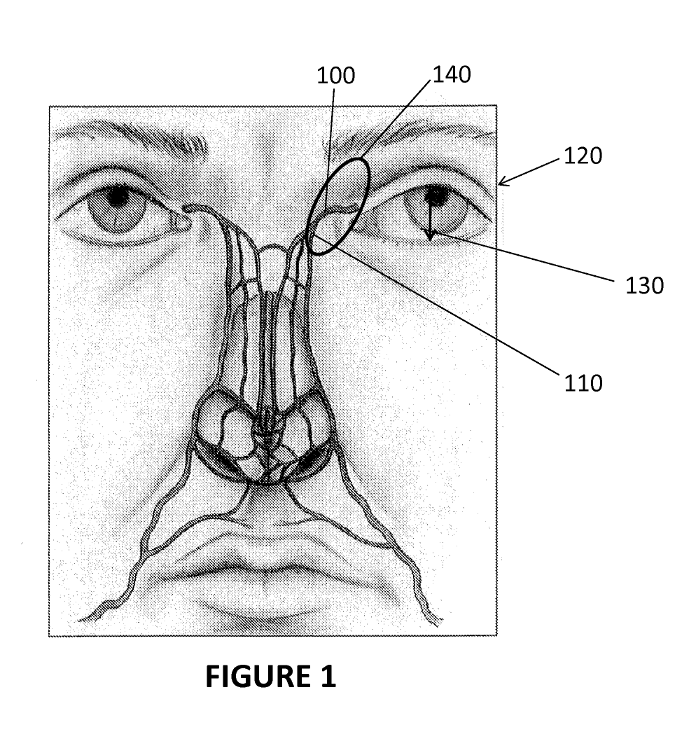

Provided according to some embodiments of the invention are photoplethysmography (PPG) sensors configured to obtain PPG signals from the ophthalmic artery region using sensors secured to the facial skin overlying or adjacent to the ophthalmic artery, its branches in the orbital area of the eye (also referred to as the dorsal nasal artery) and/or the upper third of the angular artery. As shown in FIG. 1, the ophthalmic artery 100 perforates the skull at the orbit medial to the eye 120 superomedial to the palpebral fissure 130 and anastomoses with the angular artery 110. The ophthalmic artery is directly fed by the internal carotid artery and thus PPG at this site may provide specific information regarding the blood flow to the brain. As used herein, the term “ophthalmic artery region” refers to the skin overlying the ophthalmic artery (including the branches in the orbital area of the eye, e.g., dorsal nasal artery) and the upper third of the angular artery. The ophthalmic artery region does not include the forehead, the bridge of the nose or the eye itself. FIG. 1 provides a general indication of the location of the ophthalmic artery region 140.

Thus, in some embodiments of the invention, provided are PPG sensors configured to secure to at least a portion of skin overlying or adjacent to the ophthalmic artery region. The PPG sensors are typically reflectance PPG sensors and so an emitter and detector are secured on the same surface of the skin, often close or adjacent to one another. While the sensor may be secured to skin directly overlying the ophthalmic artery region, the sensor may also be secured to skin adjacent to the skin overlying this region when the emitter and/or detector are secured at such an angle that the blood flow at in the ophthalmic artery region is monitored. Thus, as used herein, when referring to a sensor being secured to skin overlying the ophthalmic artery region, it is meant to refer to a sensor secured to skin directly overlying the ophthalmic artery region and to skin adjacent thereto when the sensor is configured to monitor blood at the ophthalmic artery region through the skin.

The term “secure” means to attach sufficiently to the skin to allow for a suitable PPG signal to be generated. In some cases, the sensor body is configured to secure onto the skin such that no additional support is necessary to allow for a suitable PPG signal to be reliably generated. However, in some cases, the sensor may be secured with the aid of an external support, for example, an additional structural support, a wire or cord, or an adhesive product such as tape. Such supports may be desirable to stabilize the sensor to prevent against signal loss, for example, due to the patient's movement (e.g., shivering), or due to movement (e.g., jostling, pulling, pushing) of the sensor or a cable attached thereto.

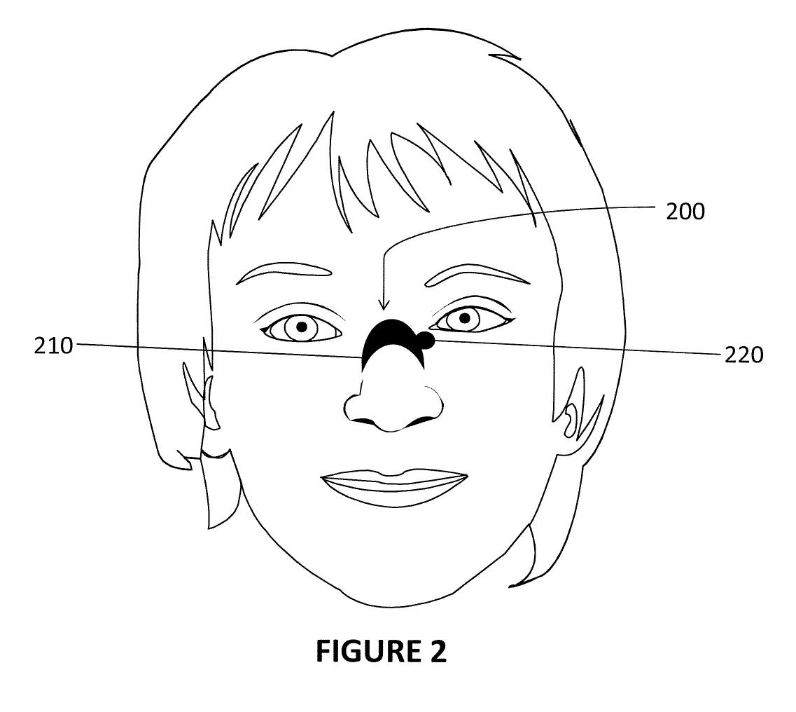

In some embodiments of the invention, a PPG sensor may be configured to secure to the nose (e.g., the nasal bridge) of the individual. The nose may provide an anchor for the sensor and provide stability so that the sensor may remain secured at the correct location. For example, FIG. 2 shows a PPG sensor 200 that includes a body portion 210 for securing over the nasal bridge and sensor portion 220 (e.g., a reflectance sensor) attached to the body portion 210 and positioned to secure to skin overlying the ophthalmic artery region. In some cases, the body portion 210 may be a clip body portion that is configured to clip onto the nasal bridge. While FIG. 2 shows the sensor portion 220 as being separately shaped and attached to the body portion 210, this is not necessary. In some cases, the sensor portion 220 may be under, part of or continuous with the body portion 210.

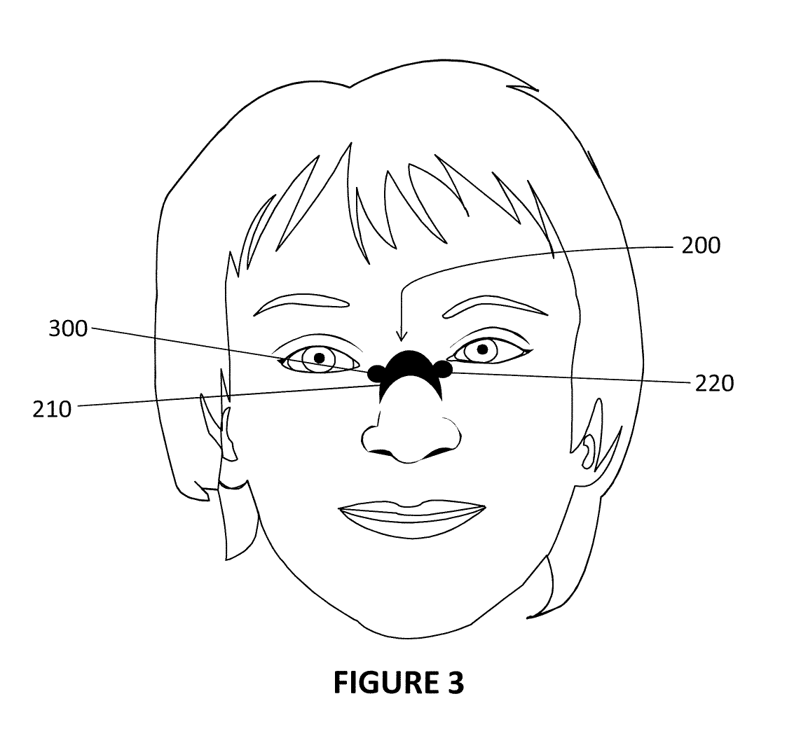

In some embodiments, the PPG sensor 200 includes at least two sensor portions so that, for example, the blood flow at both the left and the right ophthalmic artery region can be monitored. Referring to FIG. 3, the PPG sensor 200 may include a first sensor portion 220 and a second sensor portion 300. The first sensor portion 220 is configured to secure to the skin overlying the individual's left ophthalmic artery region while the second sensor portion 300 is configured to secure to the skin overlying the individual's right ophthalmic artery region. Specifically, the PPG sensors may include at least one emitter and at least one detector on skin overlying a first ophthalmic artery region and at least one emitter and at least one detector on the skin overlying a second ophthalmic artery region. Such a sensor may be useful to detect and analyze the differences between the PPG signals at the two sites to determine differences in blood flow and characteristics (e.g., blood oxygen saturation) at the two ophthalmic arteries, or the multiple signals can be used for greater confidence in parameters derived therefrom.

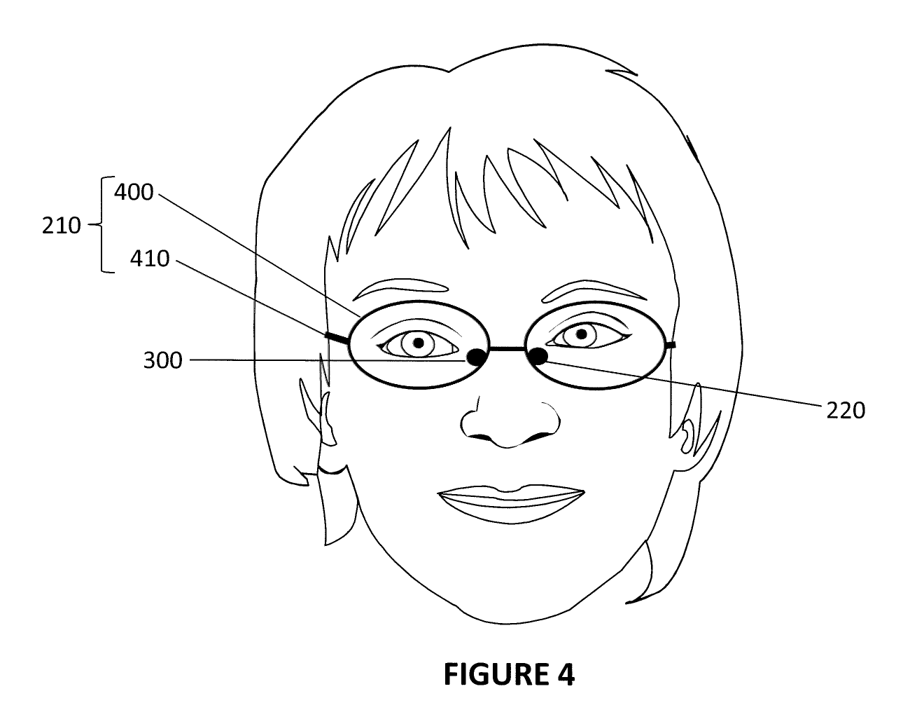

The body portion 210 of the PPG sensor 200 may be in any suitable configuration, and may include, in some cases, additional arms or support structures to the nose, forehead, ears, or other part of the face. As an example, in some embodiments, the body portion 210 of the PPG sensor 200 may be formed into an eyeglass frame, with or without lenses therein (e.g., clear or shaded lenses could be used in some cases), as shown in FIG. 4. In this case, the eyeglass frame/body portion 210 may have an eye frame portion 400 and a temple (arm) portion 410. In some cases, a sensor portion 220, maybe attached to the eye frame portion 400, and in some cases, may be attached to or integral with nose pads attached to the eye frame portion 400. Additionally a second sensor portion 300 may also be included, e.g., as attached or integral with another nose pad. In some embodiments, any wires or cabling may also be incorporated into the eyeglass frame/sensor body portion 210. In particular embodiments, wires or electronic cabling from the sensor portions, 220 and 300, may pass through the eye frame portion 400 and continue to or through one or both of the temple portions 410. The wires or cabling may then pass to a monitor or other processing device. In some cases, the eye frame portion 400 and/or the temple portion(s) 410 of the body portion 210 may include additional analysis or processing components therein. In some embodiments, the eye frame portion 400 and/or the temple portion(s) 410 of the body portion 210 may include wireless transmitters for transmitting the PPG signals or parameters derived therefrom to a processing/analysis device. In some embodiments of the invention, the body portion 210 may be connected with a band/strap for facilitating securing of the body portion 210 to the individual. For example, an elastic strap may be secured to the temple portion(s) 400 of the body portion 210 around the back of the neck or occiput to hold the frame in place. Alternatively, the body portion 210 may have a strap built into it and so the strap is part of the body portion 210.

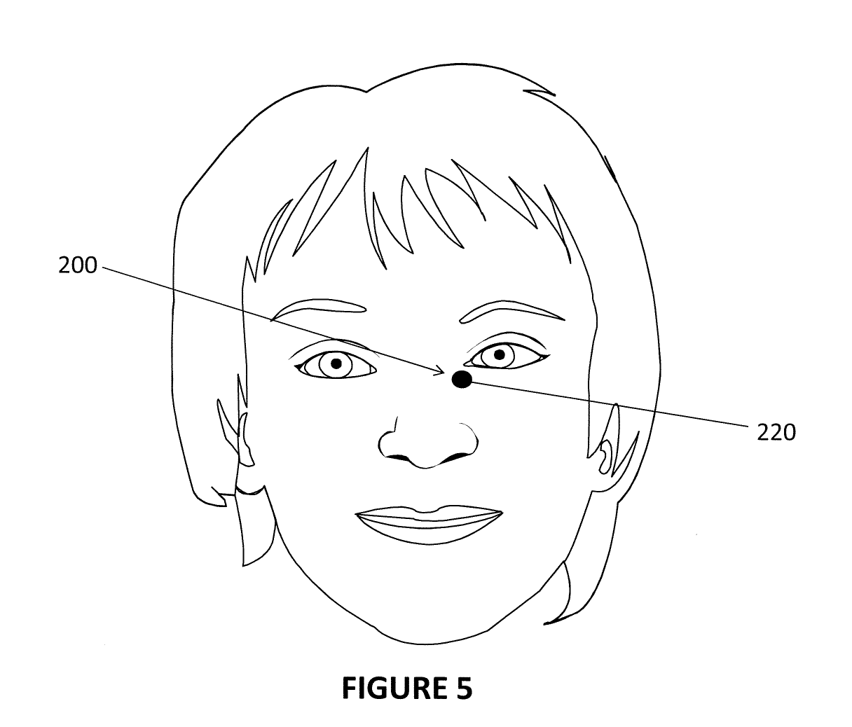

While a body portion 210 may be desirable for stability of the sensor placement, in some cases, the PPG sensor 200 may not have a body portion, or the body portion may not anchor elsewhere on the individual's face. Referring to FIG. 5, in some embodiments, the sensor portion 220 of the PPG sensor 200 may be secured onto skin overlying the ophthalmic artery region by other means, such as via an adhesive.

The body portion may be formed of and include any suitable material, including but not limited to, metals, polymers, polymer blends, and combinations thereof. Many thermoplastic and thermoset polymers may be suitable for use in the sensor body. However, in particular embodiments, the sensor body includes polycarbonate, acetal, nylon, polyester, or a combination thereof. Many metals may also be suitable for use in the sensor body, and in some embodiments, malleable metals, such aluminum and Nitinol, may be desirable. In particular embodiments, the sensor body is a molded article, such as a molded polymer article or a molded metallic article. In a particular embodiment, the PPG sensor is highly opaque and non-transmissive of light in the visible and IR spectrums to prevent the light from an emitter from reaching the detector without first passing through tissue at the measurement site.

The body portion may be composed of smaller pieces, which are assembled to form the body portion, but in some embodiments, the sensor body is a single molded article. The use of a single molded article eliminates the need for assembly of the sensor or body portion, and so may increase manufacturing efficiency and/or decrease manufacturing costs. In some embodiments, a clip body may be flexible and/or malleable. In particular embodiments, the flexural modulus of the material that forms the sensor body is in a range of 300,000 to 350,000 psi, and in some cases, in a range of 350,000 to 450,000 psi.

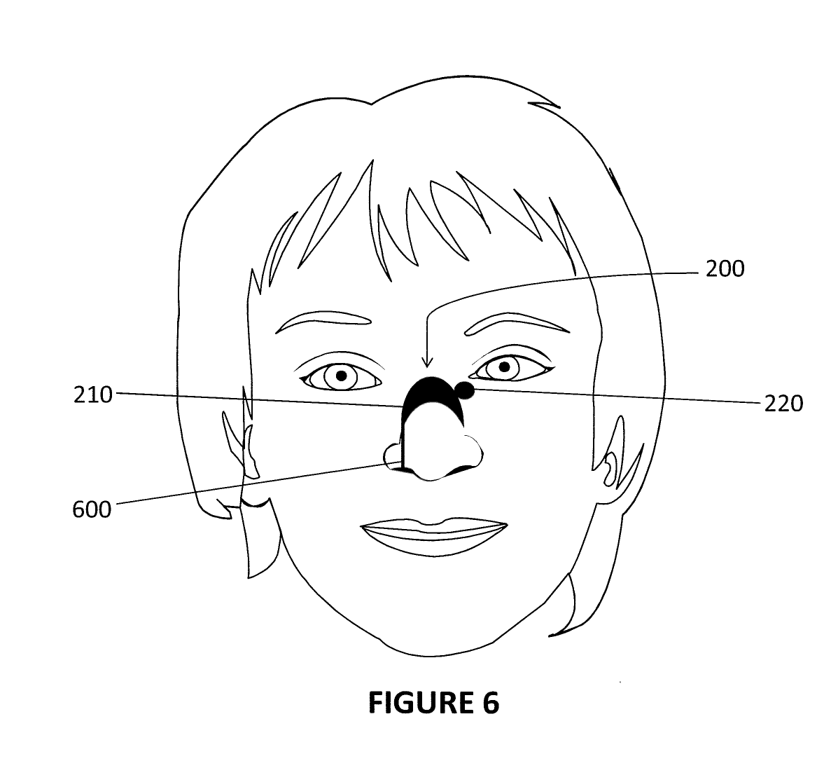

In some embodiments of the invention, the PPG sensors may include at least one additional physiological sensor, such as those used detect airflow, temperature, pressure or other respiratory parameters. Thus, in some cases, the PPG sensors described herein may include at least one secondary respiration detector, and in some embodiments, two or more secondary respiration detectors. For example, the sensor may be configured such that a first secondary respiration detector detects airflow from a first nostril, and a second secondary respiration detector detects airflow from a second nostril. FIG. 6 shows a particular example, wherein a secondary respiration detector 600 is connected to the sensor body 210 and is located in or adjacent to a nostril in order to detect air flow/pressure at the nostril. Examples of secondary respiration detectors include thermistors, thermocouples, RTDs, moisture detectors, capnometers, microphones, pressure sensors, nasal airway flow detectors, and vibration detectors.

As discussed above, the sensor portions may include at least one emitter and at least one detector, and in some cases, the PPG sensor is a reflectance sensor. As used herein, the term “light” is used generically to refer to electromagnetic radiation, and so the term includes, for example, visible, infrared and ultraviolet radiation. Any suitable type of emitter may be used, but in some embodiments, the emitter is a light-emitting diode (LED). In particular embodiments, a first emitter emits light at a first wavelength, and a second emitter emits light at a second wavelength. For example, a sensor that may be used to measure blood oxygen saturation levels may include a first emitter that emits light in the visible range and a second emitter that emits light in the infrared range. In some cases, a single emitter may emit light at a first wavelength and a second wavelength. One or more photodetectors, also referred to as “detectors,” are also included. The detector is configured to detect light from an emitter, and this detected light generates a PPG signal. Any suitable photodetector may be used. However, examples of photodetectors include photodiodes, photoresistors, phototransistors, light to digital converters, and the like.

Electronic components, including emitter(s) and detector(s), may be provided to the PPG sensors described herein by any suitable method. However, in particular embodiments, a flex circuit is used in combination with the sensor body. The flex circuit may provide at least one electronic component to the sensor, and any suitable electronic component may be included in or on the flex circuit. When a flex circuit is said to “include” or “comprise” an electronic component, it is meant that the electronic component is within the flex circuit or on a surface of the flex circuit. In order to secure sufficiently to the tissue, the flex circuit may be attached or adjacent to the sensor body. The term “attached” includes mechanical attachment, e.g., via hooks or fasteners, or chemical attachment, e.g., via adhesives. The term “adjacent” means that the flex circuit is next to and/or touching the sensor body, but not actually attached to the sensor body. As will be described in further detail below, in some embodiments, one or more elastomeric sleeves sufficiently bind the sensor body and the flex circuit together so that the sensor body and the flex circuit need not be attached to each other. Thus, in some embodiments, no adhesive is present between the flex circuit and the sensor body, between the sensor body and the elastomeric sleeve and/or between the flex circuit and the elastomeric sleeve. In some embodiments, the flex circuit could be used as the sensor body itself.

While any suitable type of flex circuit may be used, in some embodiments, the flex circuit is a single electrically conductive layer, housed in insulative plastic, which has all of the electronic components on the same side of the circuit. Furthermore, in particular embodiments, the flex circuit includes a moisture protective conformal coating.

Electronic components that provide additional physiological monitoring to the sensor may also be included on the flex circuit. As described above, in some embodiments, at least one respiration detector may be included on the sensor, and examples of respiration detectors include thermistors, thermocouples, RTDs, moisture detectors, capnometers, microphones, pressure sensors, nasal airway flow detectors, and vibration detectors. Other physiological monitoring components that may be included with the ophthalmic artery region sensors include oxygen sensors, pH sensors, and sensors for identifying and/or measuring particular compounds in the nasal airflow, ECG leads, and the like.

As described above, in some embodiments, an electronic component for wireless communication may be included in or on the ophthalmic artery region sensors, such as via the flex circuit. Any suitable wireless communication component may be included in the devices, but in some embodiments, a Bluetooth®, WiFi, Zigbee and/or infrared technology may be used. Such electronic components may communicate with a receiver apparatus so that PPG signals acquired by the sensor may be transmitted wirelessly to a control and/or signal processing unit.

In some embodiments, the PPG sensor (e.g., via the flex circuit) includes or is attached to a wire or cable for transmitting or communicating signals from the sensor to a computer or other analysis/processing equipment. In some cases, a portion of flex circuit itself may be considered part of the cabling. The flex circuit may also include a connector for coupling the flex circuit to a wire, cable or another electronic device. Any suitable wire, cable or other electrical connector may be used as the connector. In other embodiments of the invention, the PPG signals may be transmitted wirelessly, and so no wire or cabling is needed, and thus, the sensor may not include any cables or connectors.

As discussed above, in particular embodiments, the PPG sensors may include elastomeric sleeve(s). The “elastomeric sleeve” is an elastomeric material that envelops part of the sensor body and/or part of the flex circuit attached or adjacent thereto. The sleeve may be formed from more than one piece of elastomeric material, but in some embodiments, the sleeve may be a molded elastomeric sleeve, and as such, the sleeve may be a single molded elastomeric article. Different types of elastomeric sleeves are discussed in U.S. patent application Ser. No. 13/650,310, filed Oct. 10, 2012, and entitled “Photoplethysmography Sensors,” incorporated in its entirety by reference herein.

According to some embodiments of the invention, the PPG sensor is partially or completely disposable. As such, the sensor may be used for a single use or for more than one use, for example, 2-10 uses, including 2, 3, 4 or 5 uses. In such cases, the sensor body, the flex circuit and the elastomeric sleeve may be formed from a sufficiently inexpensive material that also meets safety and performance standards. In addition, the relatively few assembly steps also decrease production costs and may allow for the partial or complete disposability of the sensor. The disposability of the sensor may be advantageous in some cases because it may decrease or eliminate the need for cleaning and disinfection, which may, in turn, improve the ease of use for medical personnel. In some embodiments, only one of the sensor portion and the body portion of the PPG sensor is disposable. For example, the body portion may have a connector port therein and may be reusable, while the sensor portions are disposable and attached/detached at the connector port on the body portion.

Any suitable method of making the PPG sensors described herein may be used. However, particular methods of making some types of PPG sensors are described in U.S. patent application Ser. No. 13/650,310, filed Oct. 10, 2012, entitled “Photoplethysmography Sensors,” incorporated by reference in its entirety herein. Other methods known in the art may also be used.

The ophthalmic artery region sensors, according to particular embodiments, may also include or be combined with a nasal cannula for delivery of breathing gases, such as oxygen or oxygen-enriched air. The nasal cannula may be used separately or combined with the ophthalmic sensor to create an integral or attached device.

Also provided according to embodiments of the present invention are systems that include a PPG sensor according to an embodiment of the invention, and at least one computer configured to process and/or analyze PPG signals from the sensor. In particular embodiments, the systems are configured to perform methods described herein. For example, the systems may be configured to analyze the PPG signals to determine respiratory parameters such as respiration rate and/or respiratory effort. As another example, the PPG signals may also be analyzed to monitor blood flow at the ophthalmic artery region or blood flow to the brain.

Accessories for Use with Ophthalmic Artery Region Sensors

According to some embodiments of the present invention are earpieces that are configured to direct the flex circuit and/or other cables from the PPG sensor behind the patient's ear and so lead them away from the patient's face. The earpiece may also be configured to couple with a flex circuit, connector portion or adaptor instead of merely guide the wires or cables behind the patient's ear. In some cases, a flex circuit may be configured to directly couple with the earpiece, with or without an adaptor, and in some cases, additional wires and connectors may be included between the flex circuit and the earpiece. Earpieces that may be used with the PPG sensors according to embodiments of the present invention are discussed in U.S. patent application Ser. No. 13/650,310, filed Oct. 10, 2012, entitled “Photoplethysmography Sensors,” incorporated by reference in its entirety herein.

Also provided according to some embodiments of the invention are sensor kits. Such kits may include a PPG sensor according to an embodiment of the invention, and an applicator configured to secure the sensor. In some embodiments, the kit may include other accessories, such as an earloop, tape and/or cleaning products. The kit may also allow for the sensor, applicator, and any other accessories, to be contained within sterile packaging. The packaging, once opened, may provide a sterile sensor and applicator, and in some cases, the applicator may already be joined or attached to the sensor so that the sensor can be immediately placed on a patient. Once placed on the patient, the applicator may then be removed. Furthermore, in some embodiments, the sensor and/or applicator may be disposable so that it can be discarded after use.

### Systems and Methods for Using Ophthalmic Artery Region Sensors

The ophthalmic artery region sensors described herein may be used in any suitable fashion, and with any suitable signal processing apparatus or method. Thus, in some embodiments, provided are systems that include at least one ophthalmic artery region sensor according to an embodiment of the invention. Such systems may also include a processing apparatus, such as a computer or other analytical equipment, that is communicatingly connected to the ophthalmic artery region sensor. Examples of systems and methods that may be used in combination with the ophthalmic artery region sensors described herein may be found in U.S. Pat. No. 6,909,912, U.S. Pat. No. 7,127,278, U.S. Pat. No. 7,024,235, U.S. Pat. No. 7,785,262, U.S. Pat. No. 7,887,502, U.S. Publication No. 2008/0058621, U.S. Publication No. 2008/0190430, U.S. Publication No. 2010/0192952, PCT Application No. PCT/US2011/048083 and PCT/US2011/046943, the contents of each of which are incorporated herein by reference in their entirety.

The ophthalmic artery region sensors may be secured to the patient in any suitable manner. For example, once the ophthalmic artery region sensor is placed onto a subject, the wires/cabling may be connected to a signal processing apparatus, and signals can be generated. In embodiments wherein a wireless sensor is used, no connection of wires or cables may be necessary for use. In some cases, the sensor may be additionally secured by taping the sensor, flex circuit and/or any additional cabling. As described above, this may ensure that the sensor remains in place despite patient movement or jostling of the sensor or cables, for example, by medical personnel. In some cases, a lubricant may be applied to the ophthalmic artery region sensor or the skin to which it is to be applied to improve signal and/or to properly situate the sensor. In such cases, taping of the sensor and/or cables may also aid in securing the sensor to the patient.

In some embodiments, the ophthalmic artery region sensors may be used for determining respiration rate and/or other respiratory parameters and conditions. As such, the ophthalmic artery region PPG sensor may be used as a respiration detector. In some embodiments, the ophthalmic artery region sensors described herein may be useful with a secondary respiration detector as well, either as part of the sensor or as a separate device, to monitor respiration in a patient. The data from two or more different respiration detectors may be compared, including in real time, which may provide additional information and/or enhanced confidence of the determination of respiratory parameters. As described elsewhere herein, secondary respiration detectors include, but are not limited to, thermistors, thermocouples, RTDs, moisture detectors, capnometers, microphones, pressure sensors, nasal airway flow detectors, such as nasal flow transducers, NAP, and via detectors of vibrations in the ear.

The ophthalmic artery region sensors described herein may be used in combination with other physiological monitors as well, either as part of the sensor, if applicable, or as a separate device. Examples include oxygen sensors, pH sensors, blood pressure monitors, breath constituent monitors, blood constituent monitors, heart rate monitors, including ECG leads, and depth of anesthesia monitors.

The ophthalmic artery region sensors described herein may also be used in combination with other PPG sensors, including those designed for emplacement at the nose (e.g., nasal alar, nasal septum and bridge of the nose), lip, cheek, tongue or a selected site at the ear (e.g., ear canal, concha, pinnae, scaphoid fossa, or ear lobe), forehead, or peripheral sites (e.g., fingers and toes). Description of monitoring two or more different sites on the body can be found, for example, in U.S. Pat. No. 6,909,912, which is incorporated herein by reference in its entirety. In particular embodiments, a ophthalmic artery region sensor described herein may be used with a sensor designed for emplacement at or on the ear. Particular examples of such ear PPG probes can be found in U.S. Pat. Nos. 7,341,559; 5,551,432 and 5,673,692, and in U.S. Patent Publication Nos. 2010/0217103, 2010/0049017, 2010/0331631 and 2009/0275813, the contents of each of which is incorporated herein by reference in its entirety for this purpose. In some cases, it may be useful to place a PPG sensor at an ophthalmic artery region site and at an ear site due to the differences in blood flow at the two different sites.

In some embodiments of the present invention, an ophthalmic artery region sensor may be included in a system that provides patient feedback when certain PPG signals or certain PPG signal levels are generated. For example, when the sensor is used for respiration monitoring, the PPG sensor may be used with a system that can alert the patient when respiration appears to be irregular or depressed. In particular embodiments, once the PPG signals from the sensor indicate troubled or depressed respiration, the PPG signal processing unit communicates with a device that alerts the patient, e.g., by applying a wisp of air on the cheek (malar region) to stimulate respiration. Other methods of stimulating respiration include tickling the malar region, and application of heat, cold and/or mild electrical stimulation. In some cases, the ophthalmic artery region sensors themselves may include a mechanism for alerting the patient. For example, the sensor might include a component that provides a wisp of air to the patient's cheek or may provide mild electrical stimulation. In some embodiments, the system may also be configured to alert medical personnel or to take another appropriate action (such as reduction in opiate administration or increased supply of air to an intubated subject), at the time the stimulus is applied and/or when the stimulus does not restore the patient's breathing to acceptable levels.

In the drawings and specification, there have been disclosed embodiments of the invention and, although specific terms are employed, they are used in a generic and descriptive sense only and not for purposes of limitation, the scope of the invention being set forth in the following claims.

We claim:

1. A method for monitoring an individual, the method comprising:
securing a photoplethysmography (PPG) sensor comprising at least one emitter and at least one detector onto skin overlying an ophthalmic artery region of the individual; and
obtaining PPG signals generated by the PPG sensor.

2. The method of claim 1, further comprising analyzing the PPG signals to obtain at least one blood oxygen saturation measurement.

3. The method of claim 1, further comprising analyzing the PPG signals to monitor blood flow to the brain of the individual.

4. The method of claim 1, further comprising analyzing the PPG signals to monitor respiration in the individual.

5. The method of claim 4, wherein the PPG signals are used to monitor a respiration rate and/or a respiratory effort of the individual.

6. The method of claim 1, further comprising securing a PPG sensor comprising at least one emitter and at least one detector onto a secondary site on the individual and obtaining PPG signals generated by the PPG sensor at the secondary site on said individual.

7. The method of claim 6, wherein the secondary site is a peripheral body site.

8. The method of claim 1, wherein the PPG sensor is secured medial to the palpebral fissure of an eye of the individual.

9. The method of claim 1, wherein the PPG sensor monitors the blood at the ophthalmic artery region.

10. The method of claim 9, wherein the PPG sensor monitors the blood at the dorsal nasal artery.

11. A PPG sensor configured to secure to skin overlying the ophthalmic artery region.

12. The PPG sensor of claim 11, wherein the sensor is configured to secure to a nasal bridge.

13. The PPG sensor of claim 12, wherein the sensor comprises a body portion configured to secure to the nasal bridge and a sensor portion configured to secure to skin overlying the ophthalmic artery region.

14. The PPG sensor of claim 13, wherein the skin overlying the ophthalmic artery region is medial to a medial palpebral fissure of an eye of the individual.

15. The PPG sensor of claim 11, wherein a portion of the sensor is secured to the skin overlying the ophthalmic artery region with an adhesive.

16. The PPG sensor of claim 11, wherein the sensor is integral or attached to an eyeglass frame.

17. The PPG sensor of claim 16, wherein the sensor comprises a body portion configured as an eyeglass frame; and a sensor portion attached to the eyeglass frame and configured to secure to skin overlying the ophthalmic artery region.

18. The PPG sensor of claim 11, comprising a first sensor portion configured to secure to skin overlying a first ophthalmic artery region and a second sensor portion configured to secure to skin overlying a second ophthalmic artery region.

19. The PPG sensor of claim 11, wherein the PPG sensor monitors the blood at the ophthalmic artery region.

20. The PPG sensor of claim 19, wherein the PPG sensor monitors the blood at the dorsal nasal artery.

21. A system for PPG monitoring of an individual, comprising a PPG sensor configured to secure to skin overlying the ophthalmic artery region; and a computer configured to process and/or analyze signals from the PPG sensor.

22. The system of claim 21, wherein the system is configured to monitor respiration of the individual.

23. The system of claim 22, wherein the system is configured to determine respiration rate and/or respiratory effort of the individual.

24. The system of claim 21, wherein the PPG sensor comprises a first sensor portion configured to secure to skin overlying a first ophthalmic artery region and a second sensor portion configured to secure to skin overlying a second ophthalmic artery region.

25. The system of claim 24, wherein the system is configured to monitor a difference in blood flow between the first ophthalmic artery region and the second ophthalmic artery region.
